(19) United States
(12) Patent Application Publication
(10) Pub. No.: US 2017/0258705 A1
(43) Pub. Date: Sep. 14, 2017

(54) ROSE-PETAL BODY WASH AND PREPARATION METHOD THEREOF

(71) Applicant: Guangzhou Barburly Cosmetic Co.,Ltd., GuangZhou (CN)

(72) Inventor: Junhua Zhong, Conghua City (CN)

(21) Appl. No.: 15/067,562

(22) Filed: Mar. 11, 2016

(51) Int. Cl. A61K 8/97; A61K 8/46; A61K 8/44; A61K 8/81; A61K 8/41; A61Q 19/00; A61K 8/362; A61K 8/86; A61K 8/49; A61K 8/42; A61K 8/35; A61Q 19/10; A61K 8/34; A61K 8/19

(52) U.S. Cl. CPC A61K 8/97; A61K 8/345; A61K 8/463; A61K 8/44; A61K 8/8141; A61K 8/41; A61K 8/19; A61K 8/362; A61K 8/86; A61K 8/49; A61K 8/42; A61K 8/35; A61Q 19/10; A61Q 19/007; A61K 2800/48; A61K 2800/43

(57) ABSTRACT

A rose-petal body wash which comprises the following raw materials by mass percentage: solvent, suspending agent, thickener, humectant, odorant, neutralizer, emollient, chelating agent, preservative, color stabilizing agent, colorant, and its preparation method which comprises the following steps: (1) clean an agitator kettle completely and clean up the rose petals in advance; (2) pump in de-ionized water and start agitation, then feed partial raw materials in turn, then heat them up to 85° C. and agitate to dissolve completely, (3) after the raw materials dissolve, cool the system down to 50° C. and add NaOH solution to adjust it to a transparent solution, then add some other raw materials, (4) cool the system to 45° C., then add the remaining raw materials and adjust to an acceptable color before discharging, (5) discharge and inspect the product, if it is acceptable, store as finished product.

### BACKGROUND OF THE INVENTION

Technical Field

The present invention relates to a body wash, and specifically, to a rose-petal body wash and its preparation method.

2. Description of Related Art

The body wash is a liquid detergent used upon bathing. It can be classified into alkalic and acidic ones.

At present, there are a large variety of body washes popular in the market. Nevertheless, most of them are strong in irritation to skin and the skin after cleaning tends to be somewhat dry, not moist and soft. Moreover, such body washes have a sole function and are not good at skin caring except the function of cleaning.

### BRIEF SUMMARY OF THE INVENTION

In order to smooth away the shortcomings as above, the present invention provides a rose-petal body wash and its preparation method.

The purposes of the present invention as above will be realized through the following technical solution: a rose-petal body wash comprising the following raw materials by mass percentage:

solvent: 66.54492% water and 1% dipropylene glycol;

surfactant: 15% sodium laureth sulfate, 2.5% cocamidopropyl betaine and 3% sodium lauryl sulfate;

suspending agent: 8% acrolein/acrylic copolymer;

thickener: 1% cocamidopropyl DEA;

humectant: 1% glycine;

odorant: 1% fragrance;

neutralizer: 0.25% NaOH and 0.2% citric acid;

emollient: 0.2% rose petals and 0.01% polyethylene glycol-90M;

chelating agent: 0.2% EDTA disodium;

preservative: 0.01% methylisothiazolinone and 0.005% iodopropynyl butylcarbamate;

color stabilizing agent: 0.03% diphenyl ketone-4; and

colorant: 0.00003% carmine pigment, 0.000018% acid red pigment, 0.0000003% brilliant blue pigment and 0.00003% carbon black pigment.

A method of preparing rose-petal body wash comprising the following steps:

(1) first, clean an agitator kettle completely, then clean up the rose petals in advance and dissolve the polyethylene glycol-90M in advance;

(2) pump in de-ionized water and start agitation, feed water, sodium lauryl sulfate, acrolein/acrylic copolymer, cocamidopropyl betaine, sodium lauryl sulfate, cocamidopropyl DEA, dipropylene glycol and glycine in turn, then heat them to 85° C. and agitate to dissolve completely;

(3) after the raw materials added in Step (2) dissolve completely, cool the system to 50° C. and add NaOH solution until the system changes to a transparent solution (it is the key control point of process), then add fragrance, citric acid, rose petals, EDTA disodium, methylisothiazolinone, iodopropynyl butylcarbamate, polyethylene glycol-90M, the preprocessed petals and polyethylene glycol-90M aqueous solution;

(4) cool the system to 45° C. and add the remaining diphenyl ketone-4, carmine pigment, acid red pigment, brilliant blue pigment and carbon black pigment, adjust the color to be consistent with the color chart, visually check the appearance quality where the finished product should be a transparent paste, if the product is acceptable, discharge and weigh it, then stick on an identification card;

(5) after discharge and inspection of the product for its acceptance, can and store it as the finished product.

Said citric acid is used to adjust the acidity and the pH value of said acidity is 5.5-7.5.

The advantages of the present invention over the existing technologies lie in that: the body wash according to the present invention is low in irritation to skin and can condition and improve various skin problems in addition to cleaning function. It has the functions of skin care and enhancing the immune function of skin. With added rose petals, it is rich in amino acids and proteins, tannin, vitamins, pigments, flavonoids, polysaccharides and can function to clean, moisturize, control the oil and whiten the skin. The fragrance of rose can relieve people's emotional tension and hence relax the mode to produce a feeling of ease and happiness, eliminate the pressure and further enhance the effect of beautifying skin. Moreover, the method of preparation according to the present invention is simple in process and low in cost.

### DETAILED DESCRIPTION OF THE INVENTION

The present invention is described in detail below in conjunction with the embodiments.

A rose-petal body wash according to the present invention comprises the following raw materials by mass percentage:

solvent: 66.54492% water and 1% dipropylene glycol;

surfactant: 15% sodium laureth sulfate, 2.5% cocamidopropyl betaine and 3% sodium lauryl sulfate;

suspending agent: 8% acrolein/acrylic copolymer;

thickener: 1% cocamidopropyl DEA;

humectant: 1% glycine;

odorant: 1% fragrance;

neutralizer: 0.25% NaOH and 0.2% citric acid;

emollient: 0.2% rose petals and 0.01% polyethylene glycol-90M;

chelating agent: 0.2% EDTA disodium;

preservative: 0.01% methylisothiazolinone and 0.005% iodopropynyl butylcarbamate;

color stabilizing agent: 0.03% diphenyl ketone-4; and

colorant: 0.00003% carmine pigment, 0.000018% acid red pigment, 0.0000003% brilliant blue pigment and 0.00003% carbon black pigment.

One method of preparing rose-petal body wash comprises the following steps:

(1) first, clean an agitator kettle completely, then clean up the rose petals in advance and dissolve the polyethylene glycol-90M in advance;

(2) pump in the de-ionized water and start agitation, feed the water, sodium lauryl sulfate, acrolein/acrylic copolymer, cocamidopropyl betaine, sodium lauryl sulfate, cocamidopropyl DEA, dipropylene glycol and glycine in turn, then heat them to 85° C. and agitate to dissolve completely;

(3) after the raw materials added in Step (2) dissolve completely, cool the system to 50° C. and add NaOH solution until the system changes to a transparent solution (it is the key control point of process), then add the fragrance, citric acid, rose petals, EDTA disodium, methylisothiazolinone, iodopropynyl butylcarbamate, polyethylene glycol-90M, the preprocessed petals and polyethylene glycol-90M aqueous solution;

(4) cool the system to 45° C. and add the remaining diphenyl ketone-4, carmine pigment, acid red pigment, brilliant blue pigment and carbon black pigment, adjust the color to be consistent with the color chart, visually check the appearance quality where the finished product should be transparent paste, if the product is acceptable, discharge and weigh it, then stick on an identification card;

(5) after discharge and inspection of the product for its acceptance, can and store it as the finished product.

Said citric acid is used to adjust the acidity and the pH value of said acidity is 5.5-7.5.

The foregoing above is only the embodiment of the present invention and thus not limiting of the patent scope of the present invention. Any equivalent structure or any equivalent process variation made based on the specification and embodiments of the present invention or direct or indirect application in other related technical fields shall also be included within the scope of patent protection of the present invention.

What is claimed is:

1. A rose-petal body wash, characterized by comprising the following raw materials by mass percentage:
solvent: 66.54492% water and 1% dipropylene glycol;
surfactant: 15% sodium laureth sulfate, 2.5% cocamidopropyl betaine and 3% sodium lauryl sulfate;
suspending agent: 8% acrolein/acrylic copolymer;
thickener: 1% cocamidopropyl DEA;
humectant: 1% glycine;
odorant: 1% fragrance;
neutralizer: 0.25% NaOH and 0.2% citric acid;
emollient: 0.2% rose petals and 0.01% polyethylene glycol-90M;
chelating agent: 0.2% EDTA disodium;
preservative: 0.01% methylisothiazolinone and 0.005% iodopropynyl butylcarbamate;
color stabilizing agent: 0.03% diphenyl ketone-4; and
colorant: 0.00003% carmine pigment, 0.000018% acid red pigment, 0.0000003% brilliant blue pigment and 0.00003% carbon black pigment.

2. A method of preparing a rose-petal body wash, characterized by comprising the following steps:
(1) first, cleaning an agitator kettle completely, then cleaning up the rose petals in advance and dissolving the polyethylene glycol-90M in advance;
(2) pumping in de-ionized water and starting agitation, feeding water, sodium lauryl sulfate, acrolein/acrylic copolymer, cocamidopropyl betaine, sodium lauryl sulfate, cocamidopropyl DEA, dipropylene glycol and glycine in turn, then heating them to 85° C. and agitating to dissolve completely;
(3) after the raw materials added in Step (2) dissolve completely, cooling the system down to 50° C. and add NaOH solution until the system changes to a transparent solution, then adding fragrance, citric acid, rose petals, EDTA disodium, methylisothiazolinone, iodopropynyl butylcarbamate, polyethylene glycol-90M, the preprocessed petals and polyethylene glycol-90M aqueous solution;
(4) cooling the system to 45° C. and adding the remaining diphenyl ketone-4, carmine pigment, acid red pigment, brilliant blue pigment and carbon black pigment, adjusting its color to be consistent with the color chart, visually checking its appearance quality where the finished product should be a transparent paste, and if the product is acceptable, discharging and weighing it, then sticking on an identification card;
(5) after discharge and inspection of the product for its acceptance, canning and storing it as the finished product.

3. The method of preparing a rose-petal body wash according to claim 2, characterized in that said citric acid is used to adjust the acidity and the pH value of said acidity is 5.5-7.5.
